(19) United States
(12) Patent Application Publication
(10) Pub. No.: US 2007/0154542 A1
(43) Pub. Date: Jul. 5, 2007

(54) Oral pharmaceutical formulations containing non-steroidal anti-inflammatory drugs and acid inhibitors

(75) Inventors: James B. Tananbaum, Palo Alto, CA (US); Elizabeth Vadas, Dorval (CA); Mark A. Goldsmith, Menlo Park, CA (US)

Correspondence Address: MORGAN, LEWIS & BOCKIUS, LLP, ONE MARKET SPEAR STREET TOWER, SAN FRANCISCO, CA, 94105, US

(73) Assignee: Cogentus Pharmaceuticals, Inc., Menlo Park, CA (US)

(21) Appl. No.: 11/640,107

(22) Filed: Dec. 15, 2006

Related U.S. Application Data
(60) Provisional application No. 60/755,131, filed on Dec. 30, 2005.

(51) Int. Cl. A61K 31/5415; A61K 9/52

(52) U.S. Cl. 424/457; 514/226.2

(57) ABSTRACT

The present disclosure provides enteric coated capsules and orally dissolving films comprising non-steroidal anti-inflammatory drugs and acid inhibitors, as well as methods of treating treatment humans for pain and/or inflammation while reducing gastrointestinal side effects.

### 1. FIELD

The present teaching relates to oral pharmaceutical compositions comprising non-steroidal anti-inflammatory drugs and acid inhibitors and methods for their use in the treatment for pain and/or inflammation while reducing gastrointestinal side effects.

### 2. INTRODUCTION

Non-steroidal anti-inflammatory drugs (NSAIDs) are commonly used to treat a variety of indications including mild to moderate pain such as dental, muscular, post-operative pain, and rheumatoid arthritis, osteoarthritis, gouty arthritis and ankylosing spondylitis. Generally NSAIDs inhibit the enzyme cyclooxygenase (COX). The COX enzyme has two forms: COX-1 and COX-2. The COX-1 enzyme is the constitutive isoform and is mainly responsible for the synthesis of cytoprotective prostaglandins in the gastrointestinal tract. The COX-2 enzyme is the inducible isoform, and plays a major role in prostaglandin biosynthesis in inflammatory cells such as monocytes and macrophages. Consequently NSAIDs reduce inflammation, but at the same time, inhibit an enzyme partly responsible for protecting the gastrointestinal tract. Thus, the administration of NSAIDs are associated with gastrointestinal side effects such as perforations, ulcers and gastrointestinal bleeding. Patients in need of long term NSAID therapy often cannot receive such therapy due to its propensity to cause gastrointestinal side effects and as a result patients are deprived of beneficial NSAID therapy.

Clinical studies demonstrate an improvement in NSAID tolerability when patients are simultaneously administered acid inhibitors such as proton pump inhibitors (PPIs) or histamine H₂ receptor antagonists (H₂ blockers). Proton pump inhibitors suppress gastric acid secretion by inhibiting the H⁺, K⁺-ATPase enzyme system at the surface of the gastric parietal cell. H₂ blockers inhibit the action of histamine on stomach cells, and thus reduce stomach acid production.

Recognizing the potential benefits of administering acid inhibitors for the prevention of NSAID-induced gastrointestinal side effects, others have disclosed combining the two active agents for therapeutic purposes. Proton pump inhibiting agents are typically acid-labile compounds unstable in acidic environments such as those typically found in the stomach and are typically administered in a variety of enteric coated dosage forms. See for example U.S. Pat. Nos. 4,786,505, 6,613,354, 6,365,184, and 6,926,907. In addition, orally dissolvable films can be used to carry pharmaceutically active ingredients. See for example, U.S. Pat. No. 4,136,145; and 6,923,981. However, there remains a need for oral pharmaceutical combination formulations to reduce or eliminate the gastrointestinal side effects of NSAID therapy.

### 3. SUMMARY

The present invention relates to pharmaceutical compositions comprising one or more non-steroidal anti-inflammatory drugs (NSAIDs) and one or more acid inhibitors as active agents, termed “actives”. In some embodiments, the composition is an enteric coated capsule containing a NSAID and a proton pump inhibitor. In a specific embodiment, the composition is an enteric coated capsule containing meloxicam and omeprazole.

Another embodiment of the invention relates to pharmaceutical compositions with actives comprising one or more NSAIDs and one or more reversible proton pump inhibitors, wherein the reversible proton pump inhibitor is not enteric coated.

In another embodiment, the invention relates to orally dissolvable films including one or more non-steroidal anti-inflammatory drugs and one or more acid inhibitors as actives. In one embodiment, the actives comprise a combination of one or more NSAIDs and one or more proton pump inhibitors. In a specific embodiment, the proton pump inhibitor is enteric coated on dispersed fine particulates. In a specific embodiment, the orally dissolvable film contains meloxicam and enteric coated omeprazole. In another embodiment, the orally dissolvable film comprises a combination an NSAID and a non-enteric coated reversible proton pump inhibitor.

The pharmaceutical compositions are particularly useful in treating pain and/or inflammation in a patient. The invention also relates to methods of protecting the gastrointestinal tract from side effects associated with NSAID therapy using the compositions described herein. These and other features of the present teachings are set forth herein.

### 4. DESCRIPTION OF VARIOUS EMBODIMENTS

4.1 Definitions

As used herein, the following terms have the following meanings:

The phrase “acid inhibitor” means an agent capable of inhibiting or decreasing gastric acid secretion and includes antiulcerative compounds. The term acid inhibitor includes, but is not limited to, proton pump inhibitors, including reversible proton pump inhibitors, and H₂ blockers.

The term “NSAID” means a non-steroidal anti-inflammatory agent suitable for the treatment of pain, inflammation and/or fever.

The phrase “pharmaceutically acceptable” means moieties or compounds that are, within the scope of medical judgment, suitable for use in humans without undue toxicity, irritation, allergic response, and the like.

The phrase “therapeutically effective amount” means a sufficient quantity of NSAID and acid inhibitor which is effective in treating the targeted disorder, disease or condition, at a reasonable benefit/risk ratio.

The term “substrates” means pharmaceutically acceptable materials such as beads, particles, granules, pellets, and the like.

4.2 Compositions

4.2.1 Actives

The compositions provided herein can be administered in any dosage form suitable for oral administration. The dosage form generally comprise a combination of one or more NSAIDs and one or more acid inhibitors. Exemplary acid inhibitors include, but are not limited to proton pump inhibitors, reversible proton pump inhibitors, and H₂ blockers.

In some embodiments, the dosage form comprises one or more NSAIDs and one or more proton pump inhibitors. In some embodiments, the dosage form comprises one or more NSAIDs, one or more proton pump inhibitors and one or more H₂ blockers. In some embodiments, the dosage form comprises one or more NSAIDs and one or more H₂ blockers.

Any compound having NSAID-like activity can be used in the present dosage forms. Suitable compounds having NSAID activity include, but are non-limited to, the non-selective COX inhibitors, selective COX-2 inhibitors, selective COX-1 inhibitors, and COX-LOX inhibitors, as well as pharmaceutically acceptable salts, isomers, enantiomers, polymorphic crystal forms including the amorphous form, co-crystals, derivatives, prodrugs thereof.

Exemplary NSAIDs include, but not limited to, celecoxib (Celebrex™); rofecoxib (Vioxx™), etoricoxib (Arcoxia™), meloxicam (Mobic™), valdecoxib, diclofenac (Voltaren™, Cataflam™), etodolac (Lodine™), sulindac (Clinori™), aspirin, alclofenac, fenclofenac, diflunisal (Dolobid™), benorylate, fosfosal, salicylic acid including acetylsalicylic acid, sodium acetylsalicylic acid, calcium acetylsalicylic acid, and sodium salicylate; ibuprofen (Motrin), ketoprofen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam(Feldene™), indomethacin(Indocin™), nabumetone (Relafen™), naproxen (Naprosyn™), tolmetin, lumiracoxib, parecoxib, licofelone (ML3000), including pharmaceutically acceptable salts, isomers, enantiomers, derivatives, prodrugs, crystal polymorphs, amorphous modifications, co-crystals and combinations thereof

Any compound having acid inhibitor-like activity can be used as an acid inhibitor in the present dosage forms. One type of acid inhibitor comprises any compound having proton pump inhibitor activity. Suitable non-limiting examples of proton pump inhibitors include omeprazole (Prilosec™), esomeprazole (Nexium™), lansoprazole (Prevacid™), leminoprazole, rabeprazole (Aciphex™), and pantoprazolem (Protonix™), including pharmaceutically acceptable salts, isomers, enantiomers, derivatives, prodrugs, crystal polymorphs, amorphous modifications, co-crystals and combinations thereof.

In addition to compounds described above, the acid inhibitor may include compounds which reversibly bind to the enzyme responsible for gastric acid secretion, H⁺/K⁺ ATPase, the so called “reversible proton pump inhibitors” or “acid pump antagonists”. Suitable non-limiting examples include Sch-28080 (Schering Plough); Sch-32651 (Schering Plough), AZD-0865, AR-H047108, CS-526, pumaprazole, revaprazan (see WO 1998018784; U.S. Pat. No. 6,252,076; U.S. Pat. No. 5,990,311 and U.S. Pat. No. 5,750,531) soraprazan (see WO9605177 and WO9605199), H-335/25 (AstraZeneca) and SK&F-96067 (GlaxoSmithKline), and the reversible proton pump inhibitors disclosed, for example, in the documents U.S. Pat. No. 4,833,149, U.S. Pat. No. 5,041,442, U.S. Pat. No. 4,464,372, U.S. Pat. No. 6,132,768, including pharmaceutically acceptable salts, isomers, polymorphs, amorphous modifications, co-crystals and derivatives thereof, and combinations thereof.

Additional suitable non-limiting examples of acid inhibitors include SK&F-95601, SK&F-96067 and SK&F-97574 (GlaxoSmithKline), NC-1300 and NC-1300-B (Nippon Chemiphar); Hoe-731 (Saviprazole) (Sanofi-Aventis); IY-81149 (Ilaprazole); H-405/02 (AstraZeneca); CS-526 and R-105266 (Novartis; Sankyo;Ube); TY-11345 or nepaprazole sodium (Toa Eiyo); BY-841 (Altana Pharma), and TU-199 (TAP; Takeda), including pharmaceutically acceptable salts, isomers, polymorphs, amorphous modifications, co-crystals and derivatives thereof, and combinations thereof.

The acid inhibitor may also comprise any compound having H₂ blocker or H₂ antagonist activity. Suitable non-limiting examples include ranitidine, cimetidine, nizatidine, famotidine, as well as pharmaceutically acceptable salts, isomers, polymorphs, amorphous modifications, co-crystals, derivatives, prodrugs, enantiomers, and combinations thereof.

The oral pharmaceutical compositions described herein comprise one or more NSAIDs and one or more acid inhibitors in therapeutically effective amounts. As with other pharmaceuticals, it will be understood that the total daily usage of a pharmaceutical composition of the invention will be decided by a patient's physician. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and other factors known to those of ordinary skill in the medical arts. For example, it is well within the skill of the art to start doses of the compound at levels lower than required to achieve the desired therapeutic effect and to gradually increase the dosage until the desired effect is achieved.

The skilled artisan can adjust the amount of active ingredient in the pharmaceutical compositions or administered to a patient based upon standard techniques well known in the art. The dosage form can be administered at a dosage level up to and above conventional dosage levels for NSAIDs. General guidelines for dosing NSAIDS and acid inhibitors are know in the art. See for example U.S. Pat. Nos. 6,264,984; 6,610,701; and 6,926,907.

Suitable dosage levels will depend in part upon the effectiveness of the chosen actives and condition to be treated. Generally, the daily pharmaceutically effective amount of the compounds administered to a patient in doses typically range from about 0.1 to about 100 mg/kg body weight. In some embodiments, each dosage form will comprise 0.1-200 mg of the acid inhibitor and 0.1-1,000 mg of the NSAID(s). Preferably, each dosage form will comprise 10-80 mg of the acid inhibitor and 10-800 mg of the NSAID(s), and more preferably 10-40 mg acid inhibitor and 10-500 mg of the NSAID(s), respectively.

In some embodiment, the oral pharmaceutical composition comprises 1-500 mg of NSAID and 1-500 mg of acid inhibitor. In a specific embodiments, the oral pharmaceutical composition comprises 1-50 mg of meloxicam. and 5-100 mg of omeprazole. In some embodiment, the oral pharmaceutical composition comprises 5-50 mg of H₂ blocker.

All of the components used in the pharmaceutical compositions, including actives such as NSAIDs and acid inhibitors, or other excipients should be pharmaceutically acceptable.

In addition to the actives described herein, various additives may be added to the pharmaceutical compositions. These include, but are not limited to, pharmaceutically acceptable flavoring agents, sweeteners, stabilizing agents, preservatives, anti-microbial agents, coloring agents, antioxidants, wetting agents, surfactants, emulsifiers, efflux inhibitors and other excipients know to one skilled in the art.

Sweetening or flavoring agents, when present, may be in an amount of from 0.1 to 80% by weight based on the total weight of the composition. Suitable sweetening or flavoring agents are well known in the art. Exemplary sweetening agents include, but are not limited to, dextrose, mannitol, saccharine, sorbitol, sucrose, aspartame, or xylitol.

The pharmaceutical compositions optionally contain pharmaceutically acceptable coloring agents, water-soluble dyes or pigments, and opacifiers. Typical coloring agents include, among others, synthetic iron oxides, e.g., FD&C Red, and FD&C Blue.

In some embodiments, the pharmaceutical compositions described herein provide controlled release of one or more actives using one or more controlled release agents. The term “controlled release” is intended to mean the release of actives at a pre-selected or desired rate. This rate will vary depending upon the application. Desirable rates include fast or immediate release profiles as well as delayed, sustained or sequential release. Combinations of release patterns, such as initial release followed by lower levels of sustained release of active are specifically contemplated.

The active agent could be in the form of a powder, a liquid, a blended powder(s) with inactive agent(s), granules or pellets with or without enteric coating, a solution in a suitable solvent(s), a suspension with or without a suspending agent(s), or an emulsion with or without an emulsifier(s). The pharmaceutical compositions can be provided in various forms, such as in the form of a capsule, tablet or orally dissolvable film, in single unit dosage form, and multiunit dosage form. The pharmaceutical compositions may be provided in packets, bottles, blisters, sachets, and other types of containers, and where appropriate, accompanied by a desiccant to provide moisture protection or a device for providing a measured dose.

4.2.2 Enteric Coated Capsules

In one embodiment, an enteric coated capsule comprises one or more NSAIDs and one or more acid inhibitors. The enteric coated capsules are generally provided as orally administrable hard, soft or gel capsules, or other encapsulated dosage forms known in the art. The capsules to be enteric coated can include any of the various materials conventionally used in the pharmaceutical industry, including, by way of example and not limitation, gelatin, carrageen, polysaccharide (e.g., agar, hydroxypropyl methycellulose, hydroxyethycellulose, pectin, starch etc. or mixtures thereof). The capsule can include a plasticizer, such as glycerin, triacetin, sorbitol, polyethylene glycol, propylene glycol, citrate, and phthalate, to impart form and flexibility where desired.

The capsules are chosen to be compatible with the actives (e.g., NSAID and acid inhibitors) and with the enteric coating. In some embodiments, the capsule is enteric coated with an enteric material. Generally, the enteric material is insoluble in acid environments, such as the stomach, but is soluble in near-neutral environments such as the small intestine. Because of the enteric properties of the capsule, the capsule can pass through the stomach undissolved and the actives can be released in the intestinal tract. In some embodiments, the enteric coated capsule dissolves at a pH of between 5 and 7.5.

Various enteric materials are known in the art, a number of which are commercially available. The enteric coated capsule can be any enteric material known to those skilled in the art. The enteric materials usually comprises a polymer with enteric properties. Suitable non-limiting examples include methacrylic acid copolymers such as methacrylic acid/methyl methacrylate copolymers, methacrylic acid/ethyl acrylate copolymers, methacrylic acid/methyl acrylate/methyl methacrylate copolymers, shellac, hydroxypropyl methylcellulose phthalate, hydroxypropyl methyl cellulose acetate-succinates, hydroxypropylmethylcellulose trimellitate, cellulose acetate-phthalates, carboxymethylethylcellulose, polyvinyl acetate phthalate or a mixture of these components, or other suitable enteric polymer(s).

In some embodiments, enteric coating layer(s) can be applied using standard coating technique. The enteric coating is applied using a variety of methods known in the art, such as spraying or layering (see, e.g., U.S. Pat. No. 4,287,221). The thickness of the enteric coating is designed based on the nature of the coating material and the desired lag time or delay in release of the pharmaceutical composition. The enteric coating(s) may be applied to the capsule, or another coating, using a suitable coating technique. The enteric coating layer material may be dispersed or dissolved in either water or in suitable organic solvents for coating the capsule.

In some embodiments the enteric coating may contain effective amounts of pharmaceutically acceptable plasticizers to obtain the desired mechanical properties, such as flexibility of the enteric coating layers. Such plasticizers are, for example and without limitation, triacetin, citric acid esters, phthalic acid esters, dibutyl sebacate, cetyl alcohol, diethyl phthalate, triethyl citrate, polyethylene glycols, polysorbates or other plasticizers. The amount of plasticizer is optimized for the particular situation. The amount of plasticizer is usually above 10% by weight of the enteric coating polymer(s), preferably 15-50%, and more preferably 20-50%. Additives such as dispersants, colorants, pigments, anti-tacking agents may also be included into the enteric coating layer(s). Other compounds may be added to increase film thickness or opacity and to decrease diffusion of acidic gastric juices into the dosage form.

As will be appreciated by one skilled in the art, overcoating may be applied to the enteric coated capsule, for example, as a protective layer, flavor, and the like. Suitable overcoating materials include, but are not limited to, sugar, polyethylene glycol, polyvinylpyrrolidone, polyvinyl alcohol, polyvinyl acetate, hydroxypropyl cellulose, methylcellulose, ethylcellulose, hydroxypropyl methyl cellulose, carboxymethylcellulose sodium and the like. Additives such as plasticizers, colorants, pigments, fillers, anti-tacking and anti-static agents, such as for instance magnesium stearate, titanium dioxide, talc and other additives may also be included in the over-coating layer(s).

Several benefits are derived from the enteric coated capsules provided herein. For example, the enteric coating protects the actives, for example proton pump inhibitors, from acid degradation in the stomach. In addition, manufacturing costs can be significantly reduced and productivity increased because there is no need to enteric coat the individual active agents of the pharmaceutical compositions. Also, there is no need to enteric coat individual units of the proton pump inhibitor and formulate the enteric coated proton pump inhibitor with the other ingredients in such a way as to not compromise the integrity of the protective enteric coating. Accordingly, NSAIDs can be delivered in a enteric coated capsule with a minimum of gastrointestinal side effects typically associated with NSAIDs.

4.2.3 Non-Enteric Coated Formulations

In another embodiments, the formulation comprises a combination of one or more NSAIDs and one or more non-enteric protected acid inhibitors. Suitable formulations include, but non-limited to, capsules, tablets or films for oral administration to a subject.

The non-enteric formulations can comprise one or more NSAIDs and one or more proton pump inhibitors. Any compound having NSAID activity described herein can be used in the non-enteric coated formulations described herein.

In some embodiments, the non-enteric coated formulations comprises one or more NSAIDs and one or more reversible proton pump inhibitors or acid pump antagonists. Suitable non-limiting examples of such reversible compounds include Sch-28080 (Schering Plough); Sch-32651 (Schering Plough), AZD-0865, AR-H047108, CS-526, pumaprazole, revaprazan (see WO 1998018784; U.S. Pat. No. 6,252,076; U.S. Pat. No. 5,990,311 and U.S. Pat. No. 5,750,531) soraprazan (see WO9605177 and WO9605199), H-335/25 (AstraZeneca) and SK&F-96067 (GlaxoSmithKuine), and the reversible proton pump inhibitors disclosed, for example, in the documents U.S. Pat. No. 4,833,149, U.S. Pat. No. 5,041,442, U.S. Pat. No. 4,464,372, U.S. Pat. No. 6,132,768, including pharmaceutically acceptable salts, isomers, polymorphs, amorphous modifications, co-crystals and derivatives thereof, and combinations thereof.

In some embodiments, the non-enteric coated formulation comprises one or more NSAIDs and one or more H₂ blockers. Any of the compounds described herein having H₂ blocker activity can be used in the present formulation.

In some embodiments, the non-enteric coated formulation comprises one or more NSAIDs, one or more proton pump inhibitors and one or more H₂ blockers.

4.2.4 Orally Dissolving Films

The orally dissolving films provided herein generally comprise a combination of one or more NSAIDs and one or more acid inhibitors. It is specifically contemplated that the orally dissolving films described herein can comprise a single film layer or multiple film layers. For example, it may be desirable to form an orally dissolving film comprising a first active and a second film comprising a second active which may be layered onto the first film.

Any compound having NSAIDs activity can be used in the present formulation and suitable non-limiting examples are described herein. In some embodiments, the orally dissolving films comprises one or more NSAIDs and one or more proton pump inhibitors. Any compound having proton pump inhibitor activity can be used as the acid inhibitor in the present formulation and suitable non-limiting examples described herein.

In some embodiments, the orally dissolving films comprises one or more NSAIDs and one or more H₂ blockers. Any compound having H₂ blocker activity can be used in the present formulation and suitable non-limiting examples are described herein. In some embodiments, the orally dissolving films comprise one or more NSAIDs and one or more H₂ blockers, wherein the H₂ blocker is not enteric coated.

In some embodiments, the orally dissolving films comprises one or more NSAIDs and one or more reversible proton pump inhibitors. Any compound having reversible proton pump inhibitors activity can be used in the present formulation and suitable non-limiting examples are described herein. In some embodiments, the orally dissolving films comprises one or more NSAIDs and one or more reversible proton pump inhibitors, wherein the reversible proton pump inhibitors is not enteric coated.

Orally dissolving films and methods for making such films are well know in the art. See for example, the following references each of which is hereby incorporated by references in its entirety: U.S. Pat. Nos. 4,136,145; 4,713,243; 5,166,233; 5,700,478; 5,800,832, 5,948,430; 6,419,903, 6,177,096; 6,284,264; 6,596,298; 6,656,493; 6,709,671; 6,824,829; 6,923,981, and United States Patent Application Publication Nos.: US 2001/0046511; US 2001/0022964; US 2002/0131990; US 2003/0107149; US 2004/0151756, US 2004/0241242; US 2004/0247649; US 2004/0258896; US 2005/0184427; US 2005/0196358; US 2005/0075432 and US 2005/0037055.

Generally, the orally dissolving film can be prepared as described in U.S. Pat. No. 6,709,671; polyalcohol, surfactants, plasticizers, and possible other ingredients except the water-soluble or water-dispersible polymer(s) are dissolved in a sufficient amount of a solvent which is compatible with them. Examples of compatible solvents include water, alcohols or mixtures thereof After a clear solution has been formed, the water-dispersible polymer or mixture of water-dispersible polymers is slowly added with stirring, and heat if necessary, until a clear and homogeneous solution has been formed, followed by the addition of actives and flavors. The solution is coated onto a suitable carrier material and dried to form a film.

In some embodiments, the orally dissolving film can be prepared as described in U.S. patent publication no: 20050184427. Briefly, the desired components are combined to form a multi-component matrix, including the polymer, water, and an active or other components as desired, and the combination is formed into a sheet or film, by any method known in the art such as extrusion, coating, spreading, casting or drawing the multi-component matrix. If a multi-layered film is desired, this may be accomplished by co-extruding more than one combination of components which may be of the same or different composition. A multi-layered film may also be achieved by coating, spreading, or casting a combination onto an already formed film layer.

As described above, the desired actives can be mixed with the film forming solution to form the desired orally dissolving film. The actives can be uniformly dispersed in the film forming solution in the form of insoluble solid particles together an/or soluble actives. In some embodiments NSAID and enteric coated proton pump inhibitor granules are added to the film forming solution. In other embodiments meloxicam powder and enteric coated omeprazole granules are added to the film forming solution.

In some embodiments, NSAIDs maybe added to the film forming polymer solutions in the form of granules together with the enteric coated proton pump inhibitors granules. In some embodiments, meloxicam maybe added to the film forming polymer solutions in the form of granules together with the enteric coated omeprazole granules.

In some embodiments, the NSAID and/or acid inhibitor may be incorporated into the film-forming mixture in liquid form, such as in solution or suspension rather than as a coating on solid particles. This is particularly useful for acid inhibitors, such as reversible proton pump inhibitors, that do not require an enteric coat.

The orally dissolving films generally, comprise one or more polymers as well as fillers as desired. Film-forming polymers are well know in the art. See for example, U.S. patent application No. Ser. 11/092217. Generally, the polymer can be water soluble, water insoluble, water swelleable or a combination thereof. In some, embodiments the polymer can include cellulose or a cellulose derivative. Suitable non-limiting examples of water soluble polymers include carboxymethyl cellulose, hydroxypropylmethyl cellulose, hydroxyethyl cellulose, hydroxypropyl cellulose, polyvinyl pyrrolidone, polyvinyl alcohol, pullulansodium aginate, polyethylene glycol, acacia gum, arabic gum, xanthan gum, tragancanth gum, guar gum, polyacrylic acid, methylmethacrylate copolymer, carboxyvinyl copolymers, starch, and combinations thereof. Suitable non-limiting examples of water insoluble polymers include cellulose acetate, hydroxypropyl ethyl cellulose, hydroxypropyl methyl cellulose, phthalateethyl cellulose, phthalate and combinations thereof.

Also provided herein are orally dissolving films comprise one or more enteric coated pharmaceutical agents. Enteric coated pharmaceutical agents and methods for making such agents are well know in the art, see for example the following references each of which is hereby incorporated by references in its entirety U.S. Pat. Nos. 4,786,505; 6,013,281; 6,365,184; 6,296,876; 6,780,435; and 6,926,907. In some embodiments, the orally dissolving film comprising one or more NSAIDs and one or more acid inhibitors, in which one or more acid inhibitors is enteric coated. In some embodiments, the acid inhibitor can be coated onto the surface of particulate substrates and overcoated with an enteric coating.

The concentration of enteric coated actives in the orally dissolving films should be suitable for therapeutic benefit without causing adverse feeling in the mouth. The amount of enteric coated actives in the orally dissolving films depends on the kind of active and is usually between 0.01 and 20% (w/w), but it can be higher if necessary to achieve the desired effect.

In some embodiments, the orally dissolving film comprising one or more NSAIDs and one or more acid inhibitors, wherein one or more of the actives is coated onto the surface of particulate substrates. In some embodiments, the orally dissolving film comprising one or more NSAIDs and one or more acid inhibitors, wherein one ore more of the actives is coated onto the surface of particulate substrates and the acid inhibitor is not enteric coated. This embodiment can be used for an acid inhibitor that does not require an enteric coating, e.g. a reversible proton pump inhibitor or H₂ blocker. In a specific embodiment, the orally dissolving film comprising one or more NSAIDs and one or more reversible proton pump inhibitors, wherein the reversible proton pump inhibitor is coated onto the surface of particulate substrates and the reversible proton pump inhibitor is not enteric coated.

In another embodiment, the orally dissolving films comprise a combination of one or more NSAIDs and one or more acid inhibitors, in which one or more acid inhibitors are enteric coated. Suitable non-limiting examples of proton pump inhibitors, that can be enteric coated, include omeprazole (Prilosec™), esomeprazole (Nexium™), lansoprazole (Prevacid™), leminoprazole, rabeprazole (Aciphex™), and pantoprazolem (Protonix™), as well as pharmaceutically acceptable salts, polymorphic crystal forms, isomers, amorphous modifications, co-crystals, derivatives, prodrugs, enantiomers, and combinations thereof.

In a specific embodiment, the orally dissolving film comprises meloxicam and enteric coated omeprazole.

Various enteric coatings are known in the art, a number of which are commercially available. The enteric coating comprises a polymer with pH dependent solubility properties. The enteric coating can be any enteric material known to those skilled in the art and may be the same type of enteric materials described above.

Enteric coating layer(s) can be applied using standard coating technique. The enteric coating is applied using a variety of methods known in the art, such as spraying or layering (see, e.g., U.S. Pat. No. 4,287,221). The thickness of the enteric coating is designed based on the nature of the coating material and the desired lag time or delay in release of the pharmaceutical composition. The enteric coating(s) may be applied to the capsule, or another coating, using a suitable coating technique. The enteric coating layer material may be dispersed or dissolved in either water or in suitable organic solvents for coating.

The enteric coating may contain effective amounts of pharmaceutically acceptable plasticizers to obtain the desired mechanical properties, such as flexibility of the enteric coating layers, of the type described above.

In some embodiments, orally dissolving films provide controlled release of one or more actives using one or more controlled release agents. The polymers in orally dissolving films may also be chosen to be the agents for controlled release of one or more pharmaceutical ingredients. In some embodiments, controlled release can be achieved by providing a substantially water insoluble film that incorporates one or more pharmaceutical ingredients that will be released from the film over time. In some embodiments, a variety of different water soluble or insoluble polymers can be used and optionally include biodegradable polymers in combination.

In some embodiments, one or more pharmaceutical ingredients employed in the present invention can be incorporated into the film in a controlled release form. For example, pharmaceutical ingredients can be coated with polymers such as ethyl cellulose or polymethacrylate.

Additional components can be incorporated into the films of the present invention include, without limitation, colorants, flavors, fragrances, mouthwash components, preservatives, sweetening agents, vitamins and combinations thereof. Additional components can include, without limitation, surfactants and plasticizers for compartmentalizing the components within the mixture; polyalcohols; and thermo-setting gels such as pectin, carageenan, and gelatin, which can help maintain the dispersion of components. Citric acid, or other suitable agent, can be added to stimulate saliva production and facilitate rapid dissolution of the film in the oral cavity, and/or provide an acidic environment for an enteric coated proton pump inhibitor.

In some embodiments, the dissolving film can be adhered to the oral cavity thereby releasing a pharmaceutically active agents, for example NSAIDs and acid inhibitors. In some embodiments, the dissolving film can be adhered to the oral cavity thereby releasing some of the pharmaceutically active agents locally in the oral cavity. For example, a dissolving film comprising a NSAID and enteric coated proton pump inhibitor, in which the NSAID is released into the oral cavity, while the enteric coated proton pump inhibitor remains insoluble in the oral cavity and stomach, but is soluble in near-neutral environments such as the small intestine.

Optionally, the formulation may contain a combination of plasticizers, surfactants, colorants, sweetening agents, flavors, flavor enhancers, and/or other excipients commonly used to modify the taste of formulations intended for application to the oral cavity.

The orally dissolving films provided herein can accommodate a wide range of amounts of the active ingredients. As understood by one skilled on the art, the amount of actives incorporated into the film depend in part on the on the type of film, polymer, surface area, and thickness of the film. In some embodiments, the amount of actives to film is between 0.01 and 50% (w/w), but it can be higher if necessary to achieve the desired effect.

The oral pharmaceutical compositions can be packaged in sealed, air and moisture resistant packages to protect the actives from exposure to the environment and from oxidation, hydrolysis, volatilization resulting from interaction with the environment. The packaged oral pharmaceutical compositions can contain a full supply of the medication typically prescribed for the intended therapy. A series of unit doses can be packaged together in accordance with the prescribed regimen or treatment, e.g., a 3-90 day supply, depending on the particular therapy.

A number of benefits are derived from orally dissolving films provided herein. For example, the oral film strip formulations can be administered without water. This method of drug administration, without the need for water, is also particularly well suited for a mobile society. The orally dissolving films provided herein can be particularly appealing to subjects with difficulty in swallowing pharmaceuticals, such as children, elderly, and also in veterinary practice. In addition, the orally dissolving films provided herein provide for an accurate dosage amount. The dosage amount can be determined by the size of the film and concentration of the active in the original polymer/water or polymer/solvent combination.

4.3 Methods

Also provided is a method for protecting the gastrointestinal tract from the detrimental effects of NSAID therapy. The oral formulations described herein can be used for treatment of almost any physiological disorders for which the pharmaceutical compounds are indicated. The formulations provided herein can be administered to any subject in need of a therapy includes without limitation, humans(male or female), companion animals, including, but not limited to dogs, cats, ferrets, birds, food-source animals, including, but not limited to cows, pigs, and sheep, and zoo animals, and other similar animal species.

The formulations provided herein can be administered to subject in need of a therapy for disorders for which NSAIDs are typically indicated such as, for example, angina, aorto-pulmonary shunt occlusion, arthritis, bursitis, cognative decline, cancer, such as esophageal cancer and colon cancer, coronary artery disease, dementia, dysmenorrhea, ischemia, inflammation, fever, gout, headache, migraine headache, musculoskelatal disorders, myocardial infarction, osteoarthritis, pain, pericarditis, rheumatoid arthritis, soft tissue injury, stroke, thrombocythemia, post-operative thromboembolism, and the like.

Certain types of pain contemplated by this invention arise from pre-operative, post-operative, and both pre- and post-operative procedures. Examples of pain that are treated by this invention thus include anogenital, minor arthritic, dental, topical, associated with an upper respiratory infection, general, joint, menstrual, mild, mild to moderate, acute musculo-skeletal, moderate to moderately severe, moderate to severe, muscular, neurogenic, obstetrical, ocular, oral mucosal and gingival, post operative, pre-operative, pre- and post-operative, severe, short term, urinary tract, and pain associated with gastric hyperacidity

The formulations according to the invention are advantageous in minimizing or avoiding gastrointestinal side-effects caused by NSAID(s), such as in a continuous treatment with NSAID(s). The formulations can be administered one to several times a day. The daily dose of the active substances varies and will depend on various factors such as the individual requirements of the patients, properties of the actives, the mode of administration and disorder.

The oral film strip formulations described herein dissolve upon contact with saliva or mucosal membrane areas, eliminating the need to wash the dose down with a liquid. Generally, the oral film strip formulations are used to orally administer to a patient a combination of one or more NSAIDs and one or more acid inhibitors.

### 5. EXAMPLES

### 5.1 Example 1

### Omeprazole and Meloxicam Enteric Coated Hard Gelatin and HPMC Capsules

Omeprazole is wet granulated with excipients, which can include, but not limited to microcrystalline cellulose, mannitol, sodium starch glycolate, sodium stearyl ftimarate and hydroxypropyl methylcellulose. The granules can be prepared by high shear granulation or by extrusion and spheronization.

Meloxicam is either wet granulated or roller compacted with excipients that include but not limited to microcrystalline cellulose, lactose, crospovidone and magnesium stearate.

The omeprazole and meloxicam granules are blended in the appropriate proportion and filled into either hard gelatin or HPM capsules using conventional capsule filling equipment.

The filled capsules are enteric coated using conventional film coating technology, such as side vented pans or fluidized bed coaters. The polymers used to enteric coat the capsules may include but are not limited to methacrylic acid copolymers, polyvinyl acetate phthalate or cellulose acetate phthalate plasticized to provide the appropriate flexibility to the film to coat the capsule with a uniform and coherent film.

### 5.2 EXAMPLE 2

### Omeprazole and Meloxicam Non-Enteric Coated Hard Gelatin and HPMC Capsules

Omeprazole is wet granulated with excipients, which include but not limited to microcrystalline cellulose, mannitol, sodium starch glycolate, sodium stearyl fumarate and hydroxypropyl methylcellulose. The granules can be prepared by high shear granulation or by extrusion and spheronization. The dried granules are sized and film coated preferably by fluidized bed coating technology using polymers of known pH dependent solubility to provide protection against chemical degradation of omeprazole in the acidic environment of the upper GI tract. These polymers may include but not limited to methacrylic acid copolymers, cellulose acetate phthalate or polyvinyl acetetate phthalate.

Meloxicam is either wet granulated or roller compacted with excipients that include but not limited to microcrystalline cellulose, lactose, crospovidone and magnesium stearate.

The enteric coated omeprazole granules and uncoated meloxicam granules are blended in the appropriate proportion and filled into hard gelatin or HPMC capsules using conventional capsule filling equipment.

Alternatively, the enteric coated omeprazole granules and uncoated meloxicam granules are blended in the appropriate proportion and with excipients that include but not limited to microcrystalline cellulose, lactose, magnesium stearate, and croscarmellose sodium and are filled into hard gelatin or HPMC capsules using conventional capsule filling equipment.

### 5.3 EXAMPLE 3

### Omeprazole and Meloxicam Orally Dissolvable Films (ODT)

The film-forming natural polymers, including but not limited to xanthan gum, pullulan and carrageenan are mixed and hydrated in purified water. An aqueous solution of wetting agent, sweetener, flavoring agent and citric acid is added to the hydrated polymers and mixed to homogeneity. The citric acid is added to stimulate saliva production and facilitate rapid dissolution of the film in the oral cavity. It also provides an acidic environment for the enteric coated omeprazole. To this film forming solution are added meloxicam powder and enteric coated omeprazole granules. The wetting agent in the film forming polymer solution aids the dispersion of the water insoluble meloxicam and the enteric coated omeprazole granules. The suspension is mixed to homogeneity, cast on a suitable carrier and dried to form a film. The dried film is cut into appropriately sized pieces to provide the required dosage of the two active medicaments.

Alternatively, meloxicam maybe added to the film forming polymer solutions in the form of granules together with the enteric coated omeprazole granules.

Alternatively, the film former maybe hydroxypropyl methylcellulose, which is hydrated in an aqueous solution of surfactant, flavoring and sweetening agents and citric acid. Glycerol is added to plasticize the film. The solution is stirred until a clear, homogeneous solution is formed. Meloxicam powder is dispersed in the film forming solution, then enteric coated omeprazole granules are mixed in, the film is cast onto a suitable support, dried and cut into appropriately sized pieces.

Alternatively the film maybe formed from a mixture of hydroxypropyl methyl cellulose and polyvinyl pirrolidone, which is hydrated in the presence of surfactant, sweetening and flavoring agents. Once a homogeneous solution is obtained the medicaments in the form of meloxicam powder or meloxicam granules and enteric coated omeprazole particles are added, the film is cast onto a suitable carrier, dried and cut to the desired size.

Alternatively, the acid inhibitor maybe an H₂ antagonist without the need for enteric coating. The medicament maybe uniformly dispersed in the film forming solution in the form of insoluble solid particles together with the particles of the NSAID. Once a homogeneous dispersion is obtained the film is cast on a suitable carrier, dried and cut into the desired size.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

All patents, patent applications, publications, and references cited herein are expressly incorporated by reference to the same extent as if each individual publication or patent application was specifically and individually indicated to be incorporated by reference.

1. An enteric coated capsule comprising a non-steroidal anti-inflammatory drug and an acid inhibitor.

2. The enteric coated capsule of claim 1, wherein said enteric coating is selected from methacrylic acid copolymers, hydroxopropyl methylcellulose phthalate, cellulose acetate phthalate and combinations thereof.

3. The enteric coated capsule of claim 1, wherein the amount of non-steroidal anti-inflammatory drug is between 1 mg and 500 mg.

4. The enteric coated capsule of claim 1, wherein the non-steroidal anti-inflammatory drug is meloxicam.

5. The enteric coated capsule of claim 4, wherein the meloxicam is at an amount between 1 mg and 50 mg.

6. The enteric coated capsule of claim 1, wherein the acid inhibitor is a proton pump inhibitor.

7. The enteric coated capsule of claim 6, wherein the amount of proton pump inhibitor is between 1 mg and 500 mg.

8. The enteric coated capsule of claim 7, wherein the acid inhibitor is omeprazole.

9. The enteric coated capsule of claim 8, wherein the wherein the omeprazole is at an amount between 1 mg and 100 mg.

10. A pharmaceutical composition comprising a non-steroidal anti-inflammatory drug and a reversible proton pump inhibitor.

11. The pharmaceutical composition of claim 10 in which said inhibitors is not enteric coated.

12. An orally dissolvable film comprising a combination of pharmaceutically active agents, wherein said agents are a non-steroidal anti-inflammatory drug and an acid inhibitor.

13. The film of claim 12, wherein the amount of non-steroidal anti-inflammatory drug is between 1 mg and 100 mg.

14. The film of claim 12, wherein the non-steroidal anti-inflammatory drug is meloxicam.

15. The film of claim 12, wherein the acid inhibitor is enteric coated.

16. The film of claim 12, wherein the acid inhibitor is not enteric coated.

17. The film of claim 12, wherein the acid inhibitor is a proton pump inhibitor.

18. The film of claim 17, wherein the amount of proton pump inhibitor is at an amount between 1 mg and 100 mg.

19. The film of claim 17, wherein the wherein the proton pump inhibitor is omeprazole.

20. The film of claim 19, wherein omeprazole is enteric coated.

21. The film of claim 20, wherein the wherein the omeprazole is at an amount between 1 mg and 100 mg.

22. The film of claim 12, wherein the acid inhibitor is an H₂ antagonist.

23. The film of claim 22, wherein the H₂ antagonist is at an amount between 5 mg and 50 mg.

24. The film of claim 22 wherein the H₂ antagonist is famotidine.

25. An orally dissolvable film comprising meloxicam and enteric coated omeprazole.

26. A method of treating pain and/or inflammation in a patient comprising administering to said patient a composition according to claim 1.

27. A method of protecting the gastrointestinal tract from side effects associated with non-steroidal anti-inflammatory drug therapy in a patient comprising administering to said patient a composition according to claim 1.

28. A method of treating pain and/or inflammation in a patient comprising administering to said patient a composition according to claim 12.

29. A method of protecting the gastrointestinal tract from side effects associated with non-steroidal anti-inflammatory drug therapy in a patient comprising administering to said patient a composition according to claim 12.
